(19) United States
(12) Patent Application Publication
(10) Pub. No.: US 2012/0240954 A1
(43) Pub. Date: Sep. 27, 2012

(54) USE OF ESTER OIL IN METHOD FOR PERMING HAIR

(76) Inventors: Winfried Emmerling, Falkensee (DE); Birgit Rautenberg-Groth, Ellerau (DE)

(21) Appl. No.: 13/487,944

(22) Filed: Jun. 4, 2012

Related U.S. Application Data
(63) Continuation of application No. PCT/EP2010/067620, filed on Nov. 17, 2010.

(30) Foreign Application Priority Data
Dec. 4, 2009 (DE) 102009047528.1

(51) Int. Cl. A45D 7/06; A61K 8/37

(52) U.S. Cl. 132/204; 424/70.2; 132/206

(57) ABSTRACT

The invention describes the use of ester oil for haircare, particularly during the reduction stage, when perming hair, to corresponding perming agents and to a method for perming hair.

### CROSS REFERENCE TO RELATED APPLICATIONS

The present application is a continuation of International Application No. PCT/EP2010/067620 filed 17 Nov. 2010, which claims priority to German Patent Application No. 10 2009 047 528.1, filed 4 Dec. 2009, both of which are incorporated herein by reference.

### BACKGROUND OF THE INVENTION

The invention relates to the use of ester oil for hair care (particularly during the reducing stage) when perming hair, to the corresponding perming agents, and to a method for perming hair.

Hair perms are carried out with a known perm method in such a way that the hair is mechanically shaped and the shaping is fixed (e.g., by rolling up onto hair curlers or papillotes or defined by using a curling iron). Before, during or after this shaping, the hair is treated with an aqueous preparation of a keratin-reducing substance and, after a contact time, is rinsed with water or an aqueous solution. Sometimes it is advantageous to support the shaping by using a heating bonnet as a heat source. In a subsequent step the hair is then treated with an aqueous preparation of an oxidizing agent. At the end of a contact time the oxidizing agent is also rinsed out of the hair and the hair is released from the mechanical shaping auxiliaries (curlers, papillotes).

Likewise, keratin-containing fibers are smoothed by using keratin-reducing and keratin-oxidizing compositions. In a corresponding method, the frizzy hair is either wrapped on rollers having a large diameter of usually more than 15 mm or the hair is combed straight under the influence of the keratin-reducing composition. Instead of a roller, it is also possible to smooth the fibers on a smoothing board. Smoothing boards are usually rectangular panels (e.g., made of plastic). Here the fiber is preferably wetted with the keratin-reducing preparation.

A further possibility for smoothing hair is smoothing with a hot iron. However, the structure of the keratin-containing fiber is modified when the hair is heat-treated during smoothing (see R. McMullen et al., J. Cosmet. Sci., 1998, 49, pp. 223-244). This modification of the fiber structure should be counteracted by appropriate measures.

Generally, known shaping methods, particularly for smoothing, have the disadvantage that the keratin-containing fibers become electrostatically charged. Moreover, shaping results in regard to the degree of shaping and the regularity of the shaping using known methods could be improved. An improvement in the degree of shaping is mostly accompanied by increased damage to the keratin-containing fibers.

The cited aqueous preparation with keratin-reducing substance is usually adjusted to an alkaline pH in order to swell the hair, thereby allowing the keratin-reducing substance to penetrate deeply into the hair. The keratin-reducing substance cleaves part of the disulfide bonds of the keratin to give SH groups, thereby loosening the peptide crosslinking, and, due to the stress on the hair by the mechanical shaping, leads to a new orientation of the keratin structure. The oxidizing agent causes the disulfide bonds to re-form, thereby fixing the keratin structure again in the predetermined shape.

Neither the swelling action of the alkali nor the keratin-cleaving action of the reducing agent is completely reversible; consequently the freshly permed or smoothed hair is very susceptible to mechanical stress. Reducing steps that are too strong (e.g., due to too high concentrations of keratin-reducing substance or too long contact times) can also lead to severe hair damage, as manifested, for example, by embrittlement or loss of luster of the hair. Additionally, often other properties such as wet and dry combability, feel, suppleness, softness, gloss and tensile strength are also unsatisfactory.

Accordingly, there is still a need to find a method of permanently shaping hair wherein the danger of unwanted side effects is avoided with great certitude and by which a maximum, permanent shaping result is simultaneously obtained, particularly involving the use of a heat source.

### SUMMARY OF THE INVENTION

It has now been surprisingly found that a significant improvement of the properties of permanently shaped (permed) hair is achieved by incorporating ester oils in the keratin-reducing agent used during the shaping method, particularly prior to a heat treatment of the hair. The hair feels smooth and neither dry nor brittle and can be combed very well. The wave strength is not impaired by the addition of the ester oil.

Accordingly, a first subject matter of the invention is the use of at least one ester oil for hair conditioning and for reducing hair damage during permanent hair shaping. In this regard, in particular, the feel of the hair and the combability are noticeably improved.

Furthermore, it is inventively preferred to use an aqueous composition comprising at least one ester oil and at least one keratin-reducing compound. Suitable aqueous compositions are the second subject matter of the invention.

### DETAILED DESCRIPTION OF THE INVENTION

Ester oils according to the invention exhibit a melting point of less than 30° C. at a pressure of 1013 bar and have at least one (optionally substituted) alkyloxycarbonyl group in the molecule.

Preferred agents according to the invention contain the ester oils in an amount of 0.1 to 3 wt %, particularly 0.2 to 1.0 wt %, more particularly 0.3 to 0.5 wt %, based on total weight of the composition.

Preferred aqueous compositions are those having at least one ester oil chosen from (C₄ to C₂₀) monocarboxylic acid esters of (C₃ to C₂₀) alcohols and/or dialkyl carbonates and/or diesters of (C₃ to C₂₀) alcohols with alpha, omega-(C₄ to C₈) carboxylic acids and/or diesters of (C₂ to C₆) diols with (C₃ to C₁₀) monocarboxylic acids.

Those agents are inventively preferred and suitable wherein the ester oil is chosen from at least one compound of Formula (I) and/or Formula (II) and/or Formula (III)

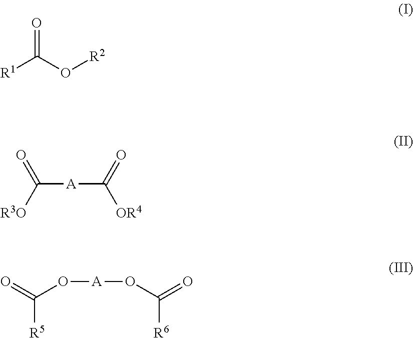

wherein
R¹ is a linear (C₃ to C₂₀) hydrocarbon group, a branched (C₃ to C₂₀) hydrocarbon group, a (C₂ to C₆) hydroxyalkyl group, a linear (C₃ to C₁₈) alkoxy group or a branched (C₃ to C₁₈) alkoxy group,
R² is a linear (C₃ to C₂₀) hydrocarbon group or a branched (C₃ to C₂₀) hydrocarbon group,
R³ and R⁴ are, independently of one another, a linear (C₃ to C₁₀) alkyl group or a branched (C₃ to C₁₀) alkyl group,
R⁵ and R⁶ are, independently of one another, a linear (C₂ to C₉) alkyl group or a branched (C₃ to C₁₀) alkyl group, and
A¹ and A² are, independently of one another, a (C₂ to C₈) alkanediyl group.

Exemplary linear (C₃ to C₂₀) hydrocarbon groups are n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-oleyl.

Exemplary branched (C₃ to C₂₀) hydrocarbon groups are isopropyl, 2-ethylhexyl, heptan-3-yl, 2-octyldodecyl, isostearyl, isocetyl.

Preferred R¹ groups according to Formula (I) are 1-hydroxyethyl, heptyl, heptan-3-yl, octan-2-yl, nonyl, undecanyl, tridecanyl, pentadecanyl, heptadecanyl.

Preferred R² groups according to Formula (I) are isopropyl, 2-ethylhexyl, octyl, decyl, dodecyl, 2-octyldodecyl, hexadecyl, isohexadecyl (i.e. isocetyl), oleyl, octadecyl or isooctadecyl (i.e. isostearyl).

Preferred R³ and R⁴ groups according to Formula (II) are independently of one another isopropyl, n-butyl, 2-ethylhexyl or n-octyl.

Preferred A¹ groups according to Formula (II) are butane-1,4-diyl, hexane-1,6-diyl or octane-1,8-diyl.

Preferred R⁵ and R⁶ groups according to Formula (III) are independently of one another heptyl, heptan-3-yl, octan-2-yl, nonyl, undecanyl, tridecanyl, pentadecanyl, heptadecanyl.

Preferred A² groups according to Formula (III) are ethane-1,2-diyl, propane-1,3-diyl, butane-1,3-diyl, butane-1,4-diyl or pentane-2,4-diyl.

The inventive effects are particularly pronounced when the agent comprises as the ester oil at least one compound chosen from 2,2-dimethylpropane-1,3-diyl di(2-ethylhexanoate) (CAS No.: 28510-23-8, e.g., Schercemol® NGDO ester from the Lubrizol company) diisopropyl adipate, di-n-butyl adipate, dioctyl maleate, diisopropyl sebacate, tridecyl neopentanoate (e.g., Ceraphyl® 55 from the ISP company), decyl oleate (e.g., Ceraphyl® 140 from the ISP company), isostearyl neopentanoate (e.g., Ceraphyl® 375 from the ISP company), isocetyl stearate (e.g., Ceraphyl® 494 from the ISP company), 2-octyldodecyl stearate (e.g., Ceraphyl®ODS from the ISP company), isopropyl myristat, isononanoic acid C₁₆₋₁₀ alkyl ester, 2-ethylhexyl palmitate, stearic acid 2-ethylhexyl ester, cetyl oleate, coco fatty alcohol caprinate/caprylate, n-butyl stearate, oleyl erucate, isopropyl palmitate, oleyl oleate, lauric acid hexyl ester, cetearyl isononanoate, lauryl lactate (e.g., Ceraphyl® 31 from the ISP company), (C12 to C15) alkyl lactate (e.g., Ceraphyl® 41 from the ISP company), dihexyl carbonate, dioctyl carbonate (e.g., Cetiol® CC from the Cognis company), didecyl carbonate.

A particularly preferred ester oil according to the present invention is chosen from at least one ester oil of the Formula (I), wherein the R¹ and R² groups are as described above and at least one of the R¹ or R² groups is a branched (C₃ to C₂₀) group. The ester oil is quite particularly preferably chosen from at least one compound of Formula (Ia)

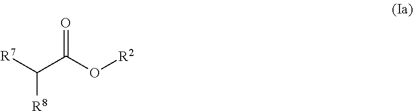

wherein
R² is a linear (C₃ to C₂₀) hydrocarbon group or a branched (C₃ to C₂₀) hydrocarbon group,
R⁷ is a (C₄ to C₆) alkyl group, and
R⁸ is a methyl group or an ethyl group.

The ester oils according to the invention are preferably used in an aqueous composition. In the context of the invention, an aqueous composition comprises at least 50 wt % water, based on total weight of the composition. This aqueous composition can be in various forms, for example, as a lotion, oil-in-water emulsion or water-in-oil emulsion.

In this regard it is again preferred to use compounds of Formula (I) during the reducing step of the permanent hair shaping (particularly as a component of an aqueous composition comprising at least one keratin-reducing compound).

The keratin-reducing compounds present in the aqueous composition are preferably chosen from compounds having at least one thiol group as well as from their derivatives, from sulfites, hydrogen sulfites and disulfites.

Exemplary compounds with at least one thiol group as well as their derivatives are thioglycolic acid, thiolactic acid, thiomalic acid, phenylthioglycolic acid, mercaptoethane sulfonic acid as well as their salts and esters (e.g., isooctyl thioglycolate and isopropyl thioglycolate), cystamine, cysteine, S-alkyl and S-aryl thiosulfates and salts of the sulfurous acids. The monoethanolammonium salts or ammonium salts of thioglycolic acid and/or of thiolactic acid as well as the free acids are preferred. They are preferably incorporated in the aqueous composition at a pH of 5 to 12, especially 7 to 9.5. In order to adjust this pH, the aqueous compositions usually contain alkalizers such as ammonia, alkali metal and ammonium carbonates and hydrogen carbonates or organic amines such as monoethanolamine.

Exemplary keratin-reducing compounds from the disulfites which can be present in the aqueous composition are alkali metal disulfites such as sodium disulfite (Na₂S₂O₅) and potassium disulfite (K₂S₂O₅), as well as magnesium disulfite and ammonium disulfite ((NH₄)₂S₂O₅). In this regard, ammonium disulfite can be inventively preferred. Exemplary keratin-reducing compounds from the hydrogen sulfites which can be present in the aqueous composition are hydrogen sulfites as an alkali metal, magnesium, ammonium or alkanolammonium salt based on a C₂-C₄ mono, di or trialkanolamine. In this regard, ammonium hydrogen sulfite can be a particularly preferred hydrogen sulfite. Exemplary keratin-reducing compounds from the sulfites which can be present in the aqueous composition are sulfites as an alkali metal, magnesium, ammonium or alkanolammonium salt based on a C₂-C₄ mono, di or trialkanolamine. Here, ammonium sulfite is preferred. When using sulfite and/or disulfite and/or hydrogen sulfite, the pH of the aqueous composition is preferably adjusted to a value in the neutral region of pH 5 to 8, preferably pH 6 to 7.5.

Inventively preferred C₂-C₄ alkanolamines are 2-aminoethanol (monoethanolamine) and N,N,N-tris(2-hydroxyethyl)amine (triethanolamine). Monoethanolamine is a particularly preferred C₂-C₄ alkanolamine that is added, particularly in an amount of 0.2 to 6 wt %, based on total aqueous composition.

The keratin-reducing compound is preferably added in an amount of 5 to 20 wt %, based on total aqueous composition.

Moreover, the aqueous composition can additionally comprise further components that promote the action of the keratin-reducing compound on the keratin. Such components include swelling agents for keratin-containing fibers such as C₁-C₆ alcohols and water-soluble glycols or polyols such as glycerin, 1,2-propylene glycol or sorbitol and urea or urea derivatives such as allantoin and guanidine, as well as imidazole and its derivatives. A preferred additional component is 1,2-propylene glycol, particularly in an amount of 0.1 to 5 wt %. The quantities refer to the total aqueous composition. In a preferred embodiment, the aqueous composition comprises 0 to 5 wt % 1,2-propylene glycol and/or 0 to 5 wt % urea.

In a preferred embodiment the inventive aqueous composition additionally comprises at least one surfactant.

Preferred surfactants are independently chosen from anionic surfactants, zwitterionic surfactants, amphoteric surfactants, non-ionic surfactants, cationic surfactants.

It is particularly preferable when agents according to the invention additionally comprise at least one non-ionic surfactant. Non-ionic surfactants contain, for example, a polyol group, a polyalkylene glycol ether group or a combination of polyol ether groups and polyglycol ether groups as the hydrophilic group. Exemplary compounds of this type are
addition products of 2 to 100 moles ethylene oxide and/or 1 to 5 moles propylene oxide to linear and branched fatty alcohols containing 8 to 30 carbon atoms, to fatty acids containing 8 to 30 carbon atoms and to alkyl phenols containing 8 to 15 carbon atoms in the alkyl group,
methyl or C₂-C₆ alkyl group end blocked addition products of 2 to 50 moles ethylene oxide and/or 1 to 5 moles propylene oxide to linear and branched fatty alcohols with 8 to 30 carbon atoms, to fatty acids with 8 to 30 carbon atoms and to alkyl phenols with 8 to 15 carbon atoms in the alkyl group, such as, for example, the commercially available types Dehydrol® LS, Dehydrol® LT (Cognis),
C₁₂-C₃₀ fatty acid mono and diesters of addition products of 1 to 30 moles ethylene oxide to glycerin,
addition products of 5 to 60 moles ethylene oxide to castor oil and hydrogenated castor oil, polyol esters of fatty acids, such as, for example, the commercial product Hydagen® HSP (Cognis) or Sovermol types (Cognis),
alkoxylated triglycerides,
alkoxylated fatty acid alkyl esters of the Formula (E4-I)

R¹CO—(OCH₂CHR²)_{w}OR³ (E4-I)
wherein R¹CO is a linear or branched, saturated and/or unsaturated acyl group containing 6 to 22 carbon atoms, R² is hydrogen or methyl, R³ is linear or branched alkyl groups containing 1 to 4 carbon atoms, and w is a number from 1 to 20,
amine oxides,
sorbitol esters of fatty acids and addition products of ethylene oxide to sorbitol esters of fatty acids such as the polysorbates,
sugar esters of fatty acids and addition products of ethylene oxide to sugar esters of fatty acids,
addition products of ethylene oxide to fatty acid alkanolamides and fatty amines,
sugar surfactants of the type alkyl and alkenyl oligoglycosides according to Formula (E4-II),

R⁴O-[G]ₚ (E4-II)
wherein R⁴ is an alkyl or alkenyl group containing 4 to 22 carbon atoms, G is a sugar group containing 5 or 6 carbon atoms, and p is a number from 1 to 10.

Amine oxides as well as addition products of 2 to 100 moles ethylene oxide and/or 1 to 5 moles propylene oxide to linear and branched fatty alcohols containing 8 to 30 carbon atoms, to fatty acids containing 8 to 30 carbon atoms and to alkyl phenols containing 8 to 15 carbon atoms in the alkyl group have proven to be quite particularly preferred non-ionic surfactants. Similarly, preparations with excellent properties are obtained when they comprise C₁₂-C₃₀ fatty acid mono and diesters of addition products of 1 to 30 moles ethylene oxide to glycerin and/or addition products of 5 to 60 moles ethylene oxide to castor oil and hydrogenated castor oil as the non-ionic surfactants.

Suitable anionic surfactants are in principle all anionic surface-active materials that are suitable for use on the human body. They are characterized by a water solubilizing anionic group such as a carboxylate, sulfate, sulfonate or phosphate group and a lipophilic alkyl group containing about 8 to 30 carbon atoms. In addition, the molecule may comprise glycol or polyglycol ether groups, ester, ether and amide groups as well as hydroxyl groups. Exemplary suitable anionic surfactants are, each in the form of the sodium, potassium and ammonium, as well as the mono, di and trialkanolammonium salts containing 2 to 4 carbon atoms in the alkanol group,
linear and branched fatty acids with 8 to 30 carbon atoms (soaps),
ether carboxylic acids of the formula R—O—(CH₂CH₂O)ₓ—CH₂—COOH, wherein R is a linear
alkyl group with 8 to 30 carbon atoms and x=0 or 1 to 16,
acyl sarcosides with 8 to 24 carbon atoms in the acyl group,
acyl taurides with 8 to 24 carbon atoms in the acyl group,
acyl isethionates with 8 to 24 carbon atoms in the acyl group,
mono- and dialkyl esters of sulfosuccinic acid with 8 to 24 carbon atoms in the alkyl group and mono-alkyl polyoxyethyl esters of sulfosuccinic acid with 8 to 24 carbon atoms in the alkyl group and 1 to 6 oxyethylene groups,
linear alkane sulfonates containing 8 to 24 carbon atoms,
linear alpha-olefin sulfonates containing 8 to 24 carbon atoms,
alpha-sulfo fatty acid methyl esters of fatty acids containing 8 to 30 carbon atoms,
alkyl sulfates and alkyl polyglycol ether sulfates of the Formula R—O(CH₂CH₂O)ₓ—OSO₃H, wherein R is preferably a linear alkyl group containing 8 to 30 carbon atoms and x=0 or 1 to 12,
mixtures of surface-active hydroxysulfonates,
sulfated hydroxyalkyl polyethylene glycol ethers and/or hydroxyalkylene propylene glycol ethers,
sulfonates of unsaturated fatty acids with 8 to 24 carbon atoms and 1 to 6 double bonds,
esters of tartaric acid and citric acid with alcohols, which represent the addition products of about 2-15 molecules of ethylene oxide and/or propylene oxide on fatty alcohols containing 8 to 22 carbon atoms,
sulfated fatty acid alkylene glycol esters of the Formula (E1-II)

R⁷CO(AlkO)ₙSO₃M (E1-II)
wherein R⁷CO— is a linear or branched, aliphatic, saturated and/or unsaturated acyl group containing 6 to 22 carbon atoms, Alk is CH₂CH₂, CHCH₃CH₂ and/or CH₂CHCH₃, n is a number from 0.5 to 5, and M is a cation, as are described in DE-OS 197 36 906,
amido ether carboxylic acids,
condensation products of C₈-C₃₀ fatty alcohols with protein hydrolysates and/or amino acids and their derivatives, known to one skilled in the art as albumin fatty acid condensates, such as the Lamepon® types, Gluadin® types, Hostapon® KCG or the Amisoft® types.

Preferred anionic surfactants are alkyl sulfates and ether carboxylic acids with 10 to 18 carbon atoms in the alkyl group and up to 12 glycol ether groups in the molecule, sulfosuccinic acid mono and dialkyl esters with 8 to 18 carbon atoms in the alkyl group and sulfosuccinic acid mono-alkyl polyoxyethyl esters with 8 to 18 carbon atoms in the alkyl group and 1 to 6 oxyethylene groups, monoglycerin disulfates, alkyl and alkenyl ether phosphates as well as albumin fatty acid condensates.

Amphoteric surfactants are understood by one skilled in the art to include the zwitterionic surfactants and ampholytic surfactants.

Zwitterionic surfactants are those surface-active compounds that carry at least one quaternary ammonium group and at least one —COO⁽⁻⁾ or —SO₃⁽⁻⁾ group in the molecule. Particularly suitable zwitterionic surfactants are betaines such as the N-alkyl-N,N-dimethylammonium glycinates, for example, the cocoalkyl dimethylammonium glycinate, N-acylaminopropyl-N,N-dimethylammonium glycinates, for example, the cocoacylaminopropyl-dimethylammonium glycinate, and 2-alkyl-3-carboxymethyl-3-hydroxyethyl imidazolines with 8 to 18 carbon atoms in each of the alkyl or acyl groups, as well as cocoacylaminoethyl hydroxyethylcarboxymethyl glycinate. A preferred zwitterionic surfactant is the fatty acid amide derivative known under the INCl name Cocamidopropyl Betaine.

Ampholytic surfactants include such surface-active compounds that, apart from a C₈₋₂₄ alkyl or acyl group, comprise at least one free amino group and at least one —COOH or —SO₃H group in the molecule and are able to form internal salts. Examples of suitable ampholytic surfactants are N-alkylglycines, N-alkylamino propionic acids, N-alkylamino butyric acids, N-alkylimino dipropionic acids, N-hydroxyethyl-N-alkylamidopropylglycine, N-alkyltaurines, N-alkylsarcosines, 2-alkylamino propionic acids and alkylamino acetic acids, each with about 8 to 24 carbon atoms in the alkyl group. Particularly preferred suitable ampholytic surfactants are chosen from N-cocoalkylamino propionate, cocoacylaminoethylamino propionate and the C₁₂-C₁₈ acyl sarcosine.

It is inventively particularly preferred if at least one surfactant of the aqueous composition is chosen from anionic surfactants, amphoteric surfactants, non-ionic surfactants.

Furthermore, it is again preferred if the aqueous composition comprises at least one non-ionic surfactant and at least one amphoteric surfactant.

The aqueous composition according to the invention preferably comprises the surfactants in an amount of 0.5 to 5.0 wt %, particularly 0.8 to 3.0 wt %, based on total weight of the aqueous composition.

The aqueous compositions can additionally comprise silicones. Inventively useable silicones are preferably linear, cyclic or branched silicones, selected from the cyclomethicones, dimethiconols, dimethicone copolyols, amodimethicones, trimethylsilylamodimethicones and phenyl trimethicones. These silicone types are known to the person skilled in the art following the nomenclature of the Cosmetic, Toiletry and Fragrance Association (CTFA) and disclosed in: M. D. Berthiaume, Society of the Cosmetic Chemists Monograph Series, “Silicones in Hair Care”, Ed.: L. D. Rhein, Ed.: Society of the Cosmetic Chemists, 1997, chapter 2, to which reference is expressly made. Polysiloxanes, such as dialkyl and alkylarylsiloxanes, for example dimethylpolysiloxane and methylphenylpolysiloxane, as well as their alkoxylated analogs, analogs terminated with hydroxyl groups and quaternized analoga, as well as cyclic siloxanes. In this regard, the silicones with the INCl names Dimethicone, PEG-12 Dimethicone, PEG/PPG-18/18 Dimethicone, Cyclomethicone, Dimethiconol, Quaternium-80 and Amodimethicone as well as their mixtures are particularly preferred.

Examples of such silicones are those products from Dow Corning commercialized with the names DC 190 (INCl name: PEG/PPG-18/18 Dimethicone), DC 193 (INCl name: PEG-12 Dimethicone), DC 200, DC1401 (INCl name: Cyclomethicone, Dimethiconol) and DC 1403 (INCl name: Dimethicone, Dimethiconol) as well as the commercial products DC 244 (INCl name: Cyclomethicone), DC 344 (INCl name: Cyclomethicone) and DC 345 (INCl name: Cyclomethicone) from Dow Corning, Q2-7224 (manufacturer: Dow Corning; a stabilized trimethylsilylamodimethicon, Dow Corning 929 Emulsion (comprising a hydroxyl-amino-modified silicone, also called Amodimethicone), SM-2059 (manufacturer: General Electric), SLM-55067 (manufacturer: Wacker) as well as Abil Quat 3270 and 3272 (manufacturer: Th. Goldschmidt; diquaternary polydimethylsiloxane, INCl name: Quaternium-80).

The silicones are preferably present in amounts of 0.1 to 10 wt %, more preferably 0.3 to 5 wt %, based on total weight of the composition.

The aqueous composition can additionally comprise protein hydrolysates. Protein hydrolysates are product mixtures obtained by acid-, base- or enzyme-catalyzed degradation of proteins (albumins). According to the invention, the added protein hydrolysates can be of vegetal as well as animal origin.

Animal protein hydrolysates include protein hydrolysates of elastin, collagen, keratin, silk and milk albumin, which can also be present in the form of their salts. Such products are marketed, for example, under the trade names Dehylan® (Cognis), Promois® (RITA Corp.), Collapuron® (Cognis), Nutrilan® (Cognis), Gelita-Sol® (Deutsche Gelatine Fabriken Stoess & Co), Lexein® (Inolex) and Kerasol® (Croda).

A preferred protein hydrolysate is the silk protein hydrolysate (Promois® Silk 720, Promois® Silk 1000).

According to the invention, it is also possible to use protein hydrolysates of vegetal origin (e.g., protein hydrolysates of soya, almond, rice, pea, potato and wheat). Such products are available, for example, under the trade names Gluadin® (Cognis), DiaMin® (Diamalt), Lexein® (Inolex) and Crotein® (Croda).

Likewise, it is possible to add derivatives of protein hydrolysates (e.g., in the form of their fatty acid condensation products). Such products are marketed, for example, under the trade names Lamepon® (Cognis), Gluadin® (Cognis), Lexein® (Inolex), Crolastin® (Croda) or Crotein® (Croda).

Although it is preferred to add the protein hydrolysates as such, optionally, other mixtures containing amino acid or individual amino acids and amino acid derivatives can also be added in their place, such as arginine, asparagine, aspartic acid, citrulline, histidine, ornithine, lysine and pyroglutamic acid. The amino acids can be added both as the free amino acid as well as the salts (e.g., as the hydrochloride or the alkali metal, alkaline earth metal or ammonium salts). Additionally, oligopeptides of on average 2-3 amino acids which have a high content (>50%, especially >70%) of the cited amino acids have also proved to be inventively useable.

Arginine, asparagine, aspartic acid as well as their salts and oligopeptides or hydrolysates, which are rich in the cited preferred amino acids, are inventively particularly preferred. Asparagine and aspartic acid as well as their salts or hydrolysates are quite particularly preferred.

Furthermore, the above cited use of ester oils or the inventive aqueous composition is suitable in particular for use during the heat-supported, permanent hair shaping—particularly at 80° C. to 250° C., more particularly at 120° C. to 200° C.

In this regard it is again preferred if a heated solid—particularly heated to 80° C. to 250° C., more particularly to 120° C. to 200° C.—as the heat source is brought in direct contact with the hair. This is particularly the case when using curling irons or smoothing irons.

Use of at least one ester oil in a heat-supported hair smoothing method is advantageous.

The preferred embodiments of the first subject matter of the invention also apply mutatis mutandis for this subject matter of the invention.

A third subject matter of the invention is a method for perming hair, wherein

(i) the hair is shaped with the aid of shaping auxiliaries after, before or during step (ii),
(ii) an aqueous composition of the second subject matter of the invention is applied onto the hair,
(iii) after a contact time Z1 the hair is rinsed and optionally dried, and
(iv) finally, an aqueous preparation comprising at least one oxidizing agent is applied on the fibers and rinsed out again after a contact time Z2.

Shaping auxiliaries include winding aids such as curlers or papillotes in the case of a perm, or auxiliaries for a mechanical smoothing such as a comb or a brush, a smoothing board or a heatable smoothing iron in the case of straightening hair. In the context of a perm method, if the shaping auxiliaries, for example, winding aids, are fixed on the fibers for a longer period of time, then these shaping auxiliaries are advisably removed after step (iv). In this connection, it can be advantageous to leave the shaping auxiliaries in the hair during step (iv), then to remove them and afterwards to repeat step (iv) as a so-called setting step (v).

The contact time Z1 is preferably 5 to 60 minutes, more preferably 10 to 30 minutes.

The contact time Z2 is preferably 1 to 30 minutes, more preferably 5 to 20 minutes.

In a preferred embodiment, the keratin-containing fibers are moistened prior to step (i). This can be accomplished by spraying the fibers with a liquid, preferably with water. Preferably, prior to step (i) the fibers are shampooed with a conventional shampoo, rinsed and then rubbed with a towel. At the conclusion of the rubbing step the hair remains noticeably damp.

The preferred suitable embodiments of the aqueous composition according to step (ii) are those cited in the second subject matter of the invention (see above).

In the context of another embodiment of the inventive method, the hair in step (iii) after rinsing or after drying, is subjected to a heat treatment—particularly at temperatures of 80° C. to 250° C., more particularly at temperatures of 120° C. to 200° C. In this regard, it is preferred if a heated solid—particularly heated to 80° C. to 250° C., more particularly to 120° C. to 200° C.—acting as the heat source is brought in direct contact with the hair. This is particularly the case when using curling irons or smoothing irons.

When using curling irons a strand of hair is wound round a suitably warmed round body (e.g., a bar-shaped or tubular body) and after a retention period—particularly 10 to 30 seconds—unwound again.

A dry keratin-containing fiber according to step (iii) of the inventive method is then present if enough of the residual water adhering to the hair is removed so that the hairs are individually separated. In a dry keratin-containing fiber, the moisture content of the fiber is preferably essentially in equilibrium with the moisture in the air or the fiber takes up moisture from the surrounding air. The drying in step (iii) is then preferably carried out if a heat treatment is effected during an additional smoothing step (e.g., with suitably warmed plates) between step (iii) and step (iv) in the context of a smoothing method.

In another development of this embodiment in the context of a hair smoothing method, the fibers in step (iii) are subjected to a heat treatment with mechanical smoothing of the fiber at a temperature of 120-220° C.

According to the invention, mechanical smoothing refers to a stretching of frizzy hair along the longest spatial dimension of the hair fiber.

Heat treatment with mechanical smoothing of the hair preferably occurs at a temperature of 140-200° C. The heat treatment can occur with hot air. In this case, during combing the hair is heated exactly at the spot where the mechanical smoothing is effected.

Moreover, the heat treatment is particularly preferably carried out in the course of smoothing with the help of suitably warmed plates, especially metallic or ceramic plates, by pressing the plate onto the hair being smoothed and moving the pressed down plate along the hair fiber. The plates can be optionally coated with heat-resistant materials. The hair fiber to be smoothed is particularly preferably pressed between two suitably warmed plates, and both plates are simultaneously moved along the longest spatial dimension of the fiber. In this regard, both plates are again preferably connected to one another, such that both plates can be evenly moved along the hair fiber. If the heat treatment is carried out on living hair, then the hair fiber is fixed at one end (hair root). In this case the plates are preferably evenly moved from the hair root along the whole of the hair fiber. This movement mechanically smoothes the fiber. A suitable instrument for the heat treatment is, for example, the “Ceramic Flat Master” instrument (commercialized by Efalock, Germany).

Furthermore, the fibers can be treated with a conventional conditioner between step (iii) and (iv) and/or in the context of a subsequent treatment at the end of the inventive method.

For carrying out the second step, the oxidative fixing, the aqueous preparation can preferably comprise, for example, hydrogen peroxide as the oxidizing agent and the usual stabilizers for stabilizing aqueous hydrogen peroxide preparations. The pH of such aqueous H₂O₂ preparations containing 0.5 to 3.0 wt % H₂O₂ is preferably in the range 2 to 4. It can be adjusted with inorganic acids, preferably phosphoric acid. Other possible oxidizing agents are sodium bromate and/or potassium bromate. Bromates of this type are added in concentrations of 1 to 10 wt % and the pH of the solution is adjusted to 4 to 7.

Apart from the oxidizing agent, additional known auxiliaries and additives can be present in the aqueous preparation for carrying out the second step of the inventive hair shaping method: They include surface-active substances, quaternary ammonium salts, cationic polymers, fragrances and opacifiers.

Both of the aqueous compositions of the inventive method can be formulated as a cream, gel or liquid. It can be preferred to make up the preparations in the form of aerosol foams with a liquefied gas such as propane-butane mixtures, N₂O, dimethyl ether, fluorohydrocarbon propellants or mixtures thereof that are filled into aerosol containers with foam valves.

The following examples are intended to describe the subject matter of the invention in more detail:

### EXAMPLES

### 1.1 Preparation of the Keratin-Reducing, Aqueous Composition

The following compositions E1 and V1 of the Table 1 were obtained by mixing the raw materials:

TABLE V

| | Quantity in wt % | Quantity in wt % |
|---|---|---|
| Raw material | E1 | V1 |
| Natrosol 250 HR | 0.25 | 0.25 |
| Ammonium thioglycolate | 12.65 | 12.65 |
| Etidronic acid | 0.15 | 0.15 |
| Ammonia (25% in water) | 1.80 | 1.80 |
| Ammonium bicarbonate | 8.80 | 8.80 |
| Plantapon ACG LC | 1.00 | 1.00 |
| Cremophor CO 60 | 2.00 | 2.00 |
| Merquat ® 100 | — | 0.10 |
| Gluadin WQ | — | 0.05 |
| Schercemol CO Ester | 0.50 | — |
| Water | ad 100 | ad 100 |

Natrosol 250 HR hydroxyethyl cellulose (INCI name: Hydroxyethylcellulose) (Hercules)
Plantapon ® ACG LC N-Cocoyl-L-glutamic acid disodium salt (31 wt % active substance in water with ca. 2.7 wt % isopropanol, INCI name: Disodium Cocoyl Glutamate) (Cognis)
Cremophor ® CO 60 PEG-60 Hydrogenated Castor Oil (BASF SE)
Merquat ® 100 poly(dimethyldiallylammonium chloride) (ca. 40% solids; INCI name: Polyquaternium-6) (Ondeo Nalco)
Schercemol ® CO Ester hexadecyl 2-ethylhexanoate (INCI name: Cetyl Ethylhexanoat; Lubrizol)
Composition E1 is inventive, composition V1 serves as a comparison.

### 1.2 Preparation of the Aqueous, Oxidizing Agent-Containing Composition

The following composition of the Table 2 was obtained by mixing the raw materials:

TABLE 2

| Raw material | Quantity in wt % |
|---|---|
| Phosphoric acid | 0.95 |
| Hydrogen peroxide | 2.00 |
| Methylparaben | 0.04 |
| Dehyquart A CA | 0.30 |
| Merquat ® 100 | 0.05 |
| Aromox MCD-W | 3.00 |
| Water | ad 100 |

Aromox ® MCD-W N,N-dimethyl-N-cocosalkylamine N-oxide (ca. 30% active substance; INCI name: Cocamine Oxide) (Akzo Nobel)
Merquat ® 100 poly(dimethyldiallylammonium chloride) (ca. 40% solids; INCI name: Polyquaternium-6) (Ondeo Nalco)
Dehyquart ® A-CA trimethylhexadecylammonium chloride (ca. 24-26% active substance; INCI name: Aqua (Water), Cetrimonium Chloride) (Cognis)

### 2. Hair Treatment

Onto each hair strand Alkinco 6634, natural dark European hair (Alkinco company) was applied per g of hair 1 ml of a composition of Table 1 and left for 20 minutes. Each strand was then rinsed for 5 minutes with water. The aqueous oxidizing agent-containing composition of Table 2 was then applied (1 ml per 1 g hair). After a contact time of 10 minutes each strand was rinsed for 5 minutes with water.

The strands were then dried in air and stored for conditioning at 25° C. and 40% relative humidity for 24 hours.

In comparison to the hair treated with composition V1, the hair treated with composition E1 exhibited a softer feel and a better combability. There was less damage to the hair. In both cases the perm result was very good.

We claim:

1. An aqueous composition comprising at least one ester oil and at least one keratin-reducing compound.

2. The aqueous composition according to claim 1, wherein the at least one ester oil is chosen from (C₄ to C₂₀) monocarboxylic acid esters of (C₃ to C₂₀) alcohols and/or dialkyl carbonates and/or diesters of (C₃ to C₂₀) alcohols with alpha, omega-(C₄ to C₈) carboxylic acids and/or diesters of (C₂ to C₆) diols with (C₃ to C₁₀) monocarboxylic acids.

3. The aqueous composition according to claim 1, wherein the at least one ester oil is chosen from at least one compound of Formula (I) and/or the Formula (II) and/or Formula (III) 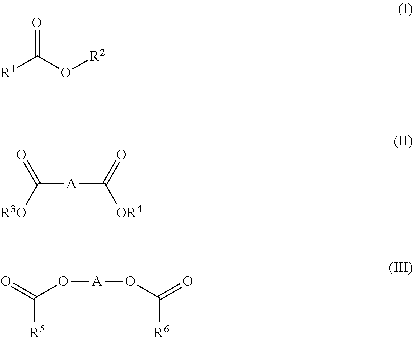
wherein
R¹ is a linear (C₃ to O₂₀) hydrocarbon group, a branched (C₃ to C₂₀) hydrocarbon group, a (C₂ to C₆) hydroxyalkyl group, a linear (C₃ to C₁₈) alkoxy group, or a branched (C₃ to C₁₈) alkoxy group,
R² is a linear (C₃ to C₂₀) hydrocarbon group or a branched (C₃ to C₂₀) hydrocarbon group,
R³ and R⁴ independently of one another are a linear (C₃ to C₁₀) alkyl group or a branched (C₃ to C₁₀) alkyl group,
R⁵ and R⁶ independently of one another are a linear (C₂ to C₉) alkyl group or a branched (C₃ to C₁₀) alkyl group, and
A¹ and A² independently of one another are a (C₂ to C₈) alkanediyl group.

4. The aqueous composition according to claim 3, wherein the ester oil is chosen from at least one ester oil of Formula (I), wherein R¹ and R² are as described above and at least one of the R¹ or R² groups is a branched (C₃ to O₂₀) hydrocarbon group.

5. The aqueous composition according to claim 1, wherein the ester oil is chosen from at least one compound of Formula (Ia) 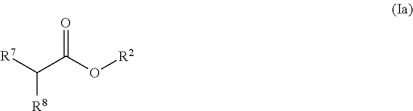
wherein
R² is a linear (C₃ to C₂₀) hydrocarbon group or a branched (C₃ to C₂₀) hydrocarbon group,
R⁷ is a (C₄ to C₆) alkyl group, and
R⁸ is a methyl group or an ethyl group.

6. The aqueous composition according to claim 1, wherein the at least one ester oil is present in an amount of 0.1 to 3.0 wt %, based on total weight of the composition.

7. The aqueous composition according to claim 1 further comprising at least one surfactant.

8. Method for perming hair comprising the steps of:
applying an aqueous composition according to claim 1 onto the hair,
shaping the hair with the aid of shaping auxiliaries before, after or during the application step,
rinsing and optionally drying the hair after a contact time Z1, and
applying an aqueous preparation comprising at least one oxidizing agent on the fibers and rinsing the preparation out after a contact time Z2.

9. The method according to claim 8 further comprising subjecting the hair to a heat treatment after the rinsing or after the optional drying after the contact time Z1.

10. The method according to claim 9 wherein the heat treatment occurs at a temperature of 80° C. to 250° C.
